(19) United States
(12) Patent Application Publication
(10) Pub. No.: US 2014/0154263 A1
(43) Pub. Date: Jun. 5, 2014

(54) Use of TAK1 Inhibitor to Achieve Glycemic Control in Mammals

(71) Applicant: Connexios Life Sciences Pvt. Ltd., Bangalore (IN)

(72) Inventors: Anup Mammen Oommen, Bangalore (IN); Mahesh Kumar Verma, Bangalore (IN); Urvashi Bahadur, Bangalore (IN); Swapnika Ramu, Bangalore (IN)

(73) Assignee: Connexios Life Sciences Pvt. Ltd., Bangalore (IN)

(21) Appl. No.: 14/097,967

(22) Filed: Dec. 5, 2013

(30) Foreign Application Priority Data
Dec. 5, 2012 (IN) 5072/CHE/2012

(51) Int. Cl. A61K 39/395

(52) U.S. Cl. CPC A61K 39/3955
USPC 424/158.1; 530/389.8

(57) ABSTRACT

The present invention relates to use of inhibitors of TAK-1 for the production of pharmaceutical agents to achieve glycemic control in mammals and which are therefore useful in the treatment of certain disorders that can be prevented or treated by inhibition of this enzyme.

### PRIORITY CLAIM

This application claims priority to provisional Indian App. No. 5072/CHE/2012 filed Dec. 5, 2012, the contents of which are incorporated by reference herein.

### FIELD OF THE INVENTION

The present invention relates to use of inhibitors of TAK-1 for the production of pharmaceutical agents to achieve glycemic control in mammals and which are therefore useful in the treatment of certain disorders that can be prevented or treated by inhibition of this enzyme. In addition, the invention relates to a novel method for the reduction in the concentration of circulating glucose by inhibitors of enzymes with similar or identical activity to the enzymatic activity of TAK-1. This invention also relates to the treatment of conditions including but not limited to Type1 Diabetes, non-insulin dependent type 2 diabetes mellitus (NIDDM), insulin resistance, obesity, impaired fasting glucose, impaired glucose and secondary complications caused due to the same.

### BACKGROUND OF THE INVENTION

Inflammation is a protective attempt by an organism to remove the injurious stimuli and to initiate the healing process. Inflammatory abnormalities are a large group of disorders which underlie a vast variety of human diseases. The immune system is often involved with inflammatory disorders, demonstrated in both allergic reactions and some myopathies.

Over the last couple of decades, an abundance of evidence has emerged demonstrating a close link between metabolism and immunity. Obesity is associated with a state of chronic low-level inflammation. The normal inflammatory response relies upon metabolic support, and energy redistribution, particularly the mobilization of stored lipid, plays an important role in fighting infection during the acute-phase response. The basic inflammatory response thus favors a catabolic state and suppresses anabolic pathways, such as the highly conserved and powerful insulin signaling pathway. The integration of metabolism and immunity, which under normal conditions is beneficial for the maintenance of good health, can become deleterious under conditions of metabolic challenge, as exemplified by the immunosuppression characteristic of malnourished or starving individuals. In the past century, however, the pendulum has also swung in the opposite direction, and now as many if not more people are overweight or obese. With the advent of this chronic metabolic overload, a new set of problems and complications at the intersection of metabolism and immunity has emerged, including the obesity-linked inflammatory diseases, diabetes, fatty liver disease, airway inflammation and atherosclerosis

Obesity is associated with a state of chronic, low-grade inflammation along with secreted adipokines which can be a causative factor for progression to diabetes. Recently European Prospective Investigation into Cancer and Nutrition (EPIC)-Potsdam study including 27,548 individuals, has shown subclinical inflammatory reaction to precede the onset of type 2 (non-insulin-dependent) diabetes.

In targeting inflammation to treat insulin resistance and diabetes, it is possible that seeking inhibitors for individual inflammatory mediators may not be a maximally effective strategy, as other redundant components may be sufficient to continue to propagate inflammatory pathways. Instead, targeting multiple inflammatory kinases is more likely to generate a robust antidiabetic action as they are known to integrate signals from multiple inflammatory mediators. Evidence from basic research suggests that stress kinases may play a significant role in integrating signals from multiple inflammatory mediators.

Several serine/threonine kinases are activated by inflammatory stimuli and contribute to inhibition of insulin signaling as well as cellular stress. This study indirectly teaches that coordinated action of both inflammatory and stress signaling can lead to loss of control on glycemia as well as loss of beta cell function progressing to diabetes and other cardiovascular and diabetic complications.

The potential kinases that will enable this desired effect include modulation of multiple kinases including JNK, P38 kinase and I Kappa B kinase. Inhibition of all these kinases together is likely to give the most desired effect. However, a person having ordinary skill in the art may find it challenging to develop a therapy that can act on all these said kinases simultaneously. Thus, the inventors of this invention believe that inhibiting a robust inflammatory kinase that has the capability of integrating several inflammatory signals with multiple stress kinases may be a very effective approach in achieving glycemic control.

Although, the connect between inflammation and diabetes have been known and understood for decades, there has been no reported attempts of modulating TAK-1 for treatment of metabolic diseases. The inventors of this invention have realized this lacuna in the science and hence have invented a method of achieving glycemic control by modulating TAK-1.

### OBJECTS OF INVENTION

The principal object of the invention is to use TAK-1 inhibitor to achieve glycemic control in mammals. Another object of the invention is to provide a method of prevention or treatment of a condition associated with TAK-1 activity in a mammal.

One other object of the invention is to provide a method for lowering elevated blood glucose level in mammals comprising administering at least one oral administration of a therapeutically effective amount of at least one TAK-1 inhibitor.

### STATEMENT OF INVENTION

The first embodiment of the present invention is a method for lowering elevated blood glucose level in mammals comprising administering at least one oral administration of a therapeutically effective amount of at least one TAK-1 inhibitor.

Another embodiment of the invention is a method where in the said at least one inhibitor is administered in combination with at least one carrier substance.

In one other embodiment the said mammals demonstrate clinically inappropriate basal, fasting and post-prandial hyperglycemia.

Another embodiment of the invention is the use of administering a therapeutically effective amount of at least one TAK-1 inhibitor to control elevated glucose level in mammals.

One other embodiment of the invention is the use of administering a therapeutically effective amount of at least one TAK-1 inhibitor for treatment of diabetes and diabetes related complications.

Another embodiment of this invention is the method for lowering elevated blood glucose levels in mammals comprising administering at least one oral administration of a therapeutically effective amount of at least one inhibitor of TAK-1 or of TAK-1 enzyme activity.

A further embodiment of the invention relates to a method of lowering elevated blood glucose levels in mammals comprising administering at least one oral administration of a therapeutically effective amount of at least one inhibitor of TAK-1 or of TAK-1 enzyme activity in combination with an adjuvant selected from and not limited to (a) dipeptidyl peptidase-IV (DP-IV) inhibitors; (b) insulin sensitizing agents; (iv) biguanides; (c) insulin and insulin mimetics; (d) sulfonylureas and other insulin secretagogues; (e) alpha.-glucosidase inhibitors; and (f) GLP-1, GLP-1 analogs, and GLP-1 receptor agonists.

In another embodiment a preferred adjuvants are biguanides more preferably metformin.

### BRIEF DESCRIPTION OF DRAWINGS

FIG. 1 shows the impact of TAK-1 inhibitor on JNK Phosphorylation under inflammatory conditions in Adipocytes.

FIG. 2 shows the impact of TAK-1 inhibition on insulin sensitivity in adipocytes.

FIG. 3 shows the impact of TAK-1 inhibition on adipogenesis in adipocytes.

FIG. 4 shows the impact of TAK-1 inhibition on secreted adiponectin in adipocytes.

FIG. 5 shows the impact of TAK-1 inhibition on secreted IL-6 levels in adipocytes.

FIG. 6 shows the impact of TAK-1 inhibition on expression of inflammation-related markers in adipocytes.

FIG. 7 shows the impact of TAK-1 inhibition on JNK phosphorylation in human hepatoma HepG2 cells.

FIG. 8 shows the impact of TAK-1 inhibition on reactive oxygen species (ROS) in rat primary hepatocytes.

FIG. 9 shows the impact of TAK-1 inhibition on insulin sensitivity in primary hepatocytes as assessed by insulin-mediated repression of gluconeogenesis.

FIG. 10 shows the impact of TAK-1 inhibition on inflammatory cytokine gene expression (MCP1 and IL1beta) in rat primary hepatocytes.

FIG. 11 shows the impact of TAK-1 inhibition on triglyceride (TAG) accumulation in human hepatoma HepG2 cells.

FIG. 12 shows expression of TAK-1 in skeletal muscle cells under diabetic condition.

FIG. 13 shows the impact of TAK-1 inhibition on inflammatory signals in skeletal muscle cells.

FIG. 14 shows the impact of TAK-1 inhibition on IkB level in skeletal muscle cells.

FIG. 15 shows the impact of TAK-1 inhibition on inflammatory cytokines gene expression in skeletal muscle cells.

FIG. 16 shows impact of TAK-1 inhibitor on muscle proteolysis.

FIG. 17 shows impact of TAK-1 inhibitor on muscle cellular stress.

FIG. 18 shows impact of TAK-1 inhibition on mitochondrial biogenesis and mitochondrial activity in skeletal muscle cells.

FIG. 19 shows the impact of TAK-1 inhibitor on the expression of PPARa and CPT1b under different disease conditions in skeletal muscles.

FIG. 20 shows impact of Inhibition of TAK-1 on IMTG content of skeletal muscle.

FIG. 21 shows impact of inhibition of TAK-1 on expression of PDK4 and glucose oxidation.

FIG. 22 shows impact of inhibition of TAK-1 on insulin sensitivity of skeletal muscle cells on exposure to inflammation reduced insulin stimulated AKT phosphorylation.

FIG. 23 shows impact of inhibition of TAK-1 on insulin sensitivity of skeletal muscle cells on exposure to inflammation induced serine phosphorylation of IRS1.

FIG. 24 shows impact of TAK-1 inhibition on glucogen content of skeletal muscle.

FIG. 25 shows impact of TAK-1 inhibition on GSIS under inflammatory conditions in islet beta cells.

FIG. 26 shows impact of TAK-1 inhibition on PDX1 and insulin gene expression under inflammatory conditions in islet beta cells.

FIG. 27 shows impact of TAK-1 inhibition on ID b gene expression under inflammatory conditions in islet beta cells.

FIG. 28 shows impact of TAK-1 inhibition on beta cell glucose sensing machinery under inflammatory conditions in islet beta cells.

FIG. 29 shows impact of TAK-1 inhibition on inflammatory cytokines gene expression in beta cells.

FIG. 30 shows decrease in fasting glucose levels in TAK1 knockdown mice.

FIG. 31 shows the improvement in glucose tolerance in TAK1 knockdown mice.

FIG. 32 shows the improvement in insulin sensitivity in TAK1 knockdown mice.

FIG. 33 shows the decrease in serum triglyceride levels in TAK1 knockdown mice.

### DETAILED DESCRIPTION OF THE INVENTION

The present invention aims at a new method to lower the level of blood glucose by lowering the activity of transforming growth factor-β activated kinase 1 (TAK-1) which is a key molecule in pro-inflammatory signaling pathways. TAK-1 inhibition can be expected to be effective in diseases associated with inflammation.

The term “inhibition” shall mean partial or complete lowering of activity of the enzyme or protein unless specified particularly otherwise in this document.

TAK-1 is a cystolic kinase and is a member of the mitogen-activated protein kinase kinase kinase (MAP3K) family, originally identified as a key regulator of MAP kinase activation in TGFβ/BMP signaling. It is a crucial upstream mediator of inflammatory signaling pathways. It forms a kinase complex including TRAF6, MAP3K7P1/TAB1 and MAP3K7P2/TAB2. The said complex is required for the activation of nuclear factor kappa B. It also activates MAPK8/JNK, MAP2K4/MKK4, and thus plays a role in the cell response to systemic stress.

Studies have reported that Drosophila TAK-1 is required for both c-jun N-terminal kinase and NFκB activation in response to immune challenge by gram-negative bacteria infection. TAK-1 has also been shown to function as a critical upstream molecule of NFκB and MAPK signaling in various mammalian cell types after stimulation with IL1, TNF and lipopolysaccharide, which activates Toll-like receptor (TLR) signaling.

Prior art indicates that stress kinases are key mediators of insulin resistance and beta cell apoptosis under inflammatory as well as systemic stress. In the light of this teaching, the inventors of this invention hypothesized that in a diseased condition, TAK-1 may potentially cause loss of beta cell function and glycemic control. Hence modulating TAK-1 is likely to help in alleviation of this condition. The inventors performed a series of experiments to validate the hypothesis and have surprisingly found that inhibition of TAK-1 indeed helps in improving insulin sensitivity and beta cell function.

The inventors through several experiments have shown that TAK-1 inhibition improves glycemic control through its impact on Adipocytes, beta cells, hepatocytes, skeletal muscles and macrophages. The results clearly help in validating the role of TAK-1 inhibitor in achieving glycemic control and hence its use in treatment of diabetes, prediabetes and other diabetes related secondary complications.

In the present invention, the TAK-1 inhibitor can be any agent that inhibits TAK-1 enzyme activity including but not limited inhibitors of TAK-1 selected from small molecules, nucleotide sequences, polypeptide sequences and siRNA, pseudosubstrates that may cause inhibition of TAK-1 and binding proteins or antibodies against said enzyme proteins.

The invention concerns the use of TAK-1 inhibitor for lowering of elevated glucose levels such as those found in mammals demonstrating clinically inappropriate basal, fasting and post-prandial hyperglycemia. The use according to the invention is more specifically characterized by the administration of TAK-1 inhibitors in the prevention or alleviation of pathological abnormalities of Metabolism of mammals such as hyperglycemia, low glucose tolerance, glucosuria, hyperlipidemia, hypertriglyceridemia, hypercholesterolemia, dyslipedemia, metabolic acidosis, obesity, diabetes mellitus and diabetes related secondary complications.

Accordingly compounds of the invention would be expected to have useful therapeutic properties especially in relation to prediabetic condition, insulin dependant diabetes mellitus, non insulin dependant diabetes mellitus and diabetic related secondary complications.

The term “Diabetic related secondary complication/s” shall include hyperlipidemia, hypertriglyceridemia, hypercholesterolemia, dyslipidemia, glaucoma, hypertension, atherosclerosis and its sequelae, retinopathy, nephropathy, neuropathy, osteoporosis, osteoarthritis, dementia, depression, neurodegenerative disease, psychiatric disorders, virus diseases, steatosis, fatty liver, nonalcoholic steatohepatitis, cirrhosis and inflammatory diseases

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

The method according to the present invention is a new approach to the reduction of elevated circulating glucose concentrations in the blood of mammals.

The method is simple, commercially useful and appropriate for use in therapy, especially of human diseases, which are associated with elevated or inappropriate blood glucose levels.

The TAK-1 inhibitors may be used or administered in combination with one or more additional drug(s) for the treatment of the disorder/diseases mentioned. The inhibiting agent can be administered in the same formulation or in separate formulations. If administered in separate formulations the components can be administered sequentially or simultaneously with the other drug(s).

In addition to being able to be administered in combination with one or more additional drugs, the compounds of the invention may be used in a combination therapy. When this is done the component can be typically administered in combination with each other. Thus one or more therapeutic agents may be administered either simultaneously (as a combined preparation) or sequentially in order to achieve a desired effect. This is especially desirable where the therapeutic profile of each compound is different such that the combined effect of the two drugs provides an improved therapeutic result.

The TAK-1 inhibiting agent may also be administered in combination with (or simultaneously or sequentially with) an adjuvant to increase compound performance. Suitable adjuvants may include (a) dipeptidyl peptidase-IV (DP-IV) inhibitors; (b) insulin sensitizing agents; (iv) biguanides; (c) insulin and insulin mimetics; (d) sulfonylureas and other insulin secretagogues; (e) alpha.-glucosidase inhibitors; and (f) GLP-1, GLP-1 analogs, and GLP-1 receptor agonists. The adjuvants may be part of the same composition, or the adjuvants may be administered separately (either simultaneously or sequentially). The order of the administration of the composition and the adjuvant will generally be known to the medical practitioner involved and may be varied. One of the preferred adjuvant for such administration is biguanide. The most preferred biguanide for such administration is metformin.

In one other embodiment the TAK-1 inhibitor is administered as a substitute as monotherapy or in combination, in an event of failure of treatment by agent selected from the group consisting of dipeptidyl peptidase-IV (DPP-IV) inhibitors; (b) insulin sensitizing agents; (c) insulin and insulin mimetics; (d) sulfonylureas and other insulin secretagogues; (e) alpha.-glucosidase inhibitors; (f) GLP-1, GLP-1 analogs, and GLP-1 receptor agonists; and combinations thereof.

Depending on the endogenous stability and on the bioavailability of the effectors single or multiple administrations are suitable to reach the anticipated normalization of the blood glucose concentration.

### Experimental Data:

Through a series of experiments the inventors of this invention has established the role of TAK-1 in achieving glycemic control because of its impact on adipose tissue, muscle, pancreas, hepatocytes and macrophages. The impact of TAK-1 inhibition in vitro was studied using a known TAK-1 inhibitor namely 5 (Z)-7-Oxazeanenol.

### Impact on Adipocytes:

It has been well established in prior art that Adipose tissue dysfunction plays an important role in the onset and progression of T2DM. Adipose tissue is a central store depot for energy. Excess nutrient overload causes increased adipocyte cell size due to storage of fat, resulting in hypertrophy. The subsequent release of pro-inflammatory cytokines by the adipocytes, enables macrophage recruitment and leads to an inflammatory state in the adipose tissue which impairs adipogenesis and functional adipokine profile. Increased lipolysis and secretion of inflammatory cytokines by adipose tissue lead to ectopic fat storage and insulin resistance, contributing to disease conditions. Since TAK-1 is a crucial upstream mediator of inflammatory signaling pathways the inventors hypothesized that inhibition of TAK-1 may lead to attenuation of disease pathology in adipose. This has been proved by the inventors through experiments

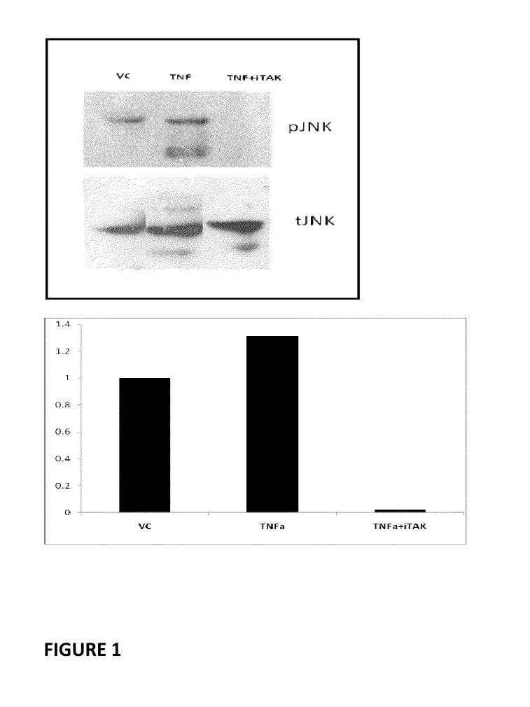

Inhibition of TAK-1 (iTAK) by small molecule inhibitor, (5Z)-7-Oxozeaenol, resulted in decreased activation of JNK in response to inflammatory cytokine (FIG. 1).

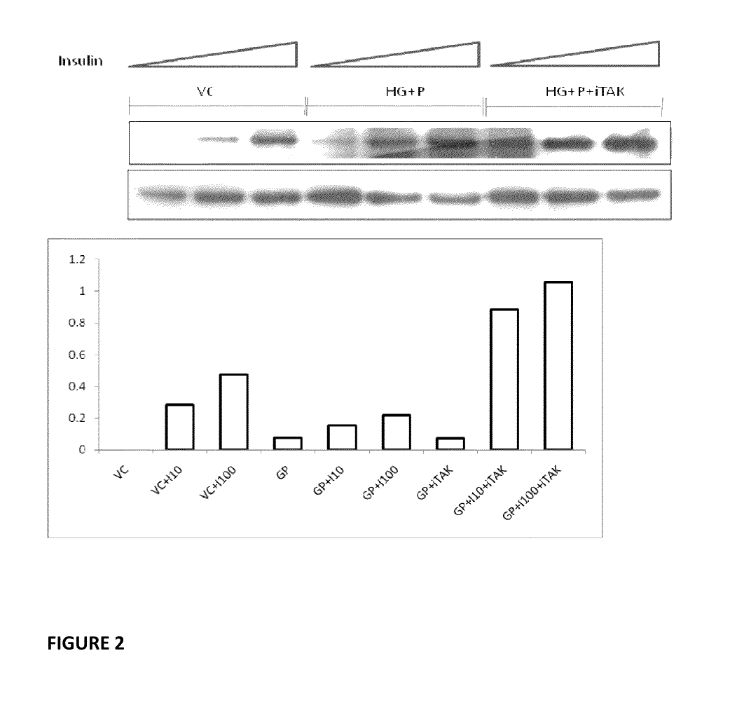

Similarly inhibition of TAK-1 improved insulin sensitivity in adipocytes under metabolic overload and inflammatory conditions (FIG. 2). This was ascertained by treating 3T3 L1 cells with glucose and palmitate (GP) for 48 hours in the presence and absence of TAK-1 inhibitor. Cells were then stimulated with 10 and 100 nM insulin (I) for 15 min, following which they were taken up for Western blot analysis for p-Akt (Thr-308) and total Akt. Chronic treatment with palmitate and inflammatory cytokines reduced responsiveness to insulin as compared to the control (VC). Inhibition of TAK-1 under such conditions, restored insulin sensitivity (P=Palmitate, iTAK=TAK-1 inhibitor). Hence it can be inferred that inhibition of TAK-1 in adipocytes has the potential to reduce inflammatory signal and increase insulin sensitivity to improve adipocyte function.

Adipogenesis is a process by which pre-adipocytes differentiate into mature, insulin sensitive, fat-laden adipocytes under hormonal influence. This process is inhibited under inflammatory conditions. Increasing adipocyte differentiation in such a context is therefore beneficial as it would allow for efficient storage of excessive nutrients and prevent hypertrophy. TAK-1 inhibition could therefore serve as a strategy for attenuating the effect of inflammation on adipocyte differentiation.

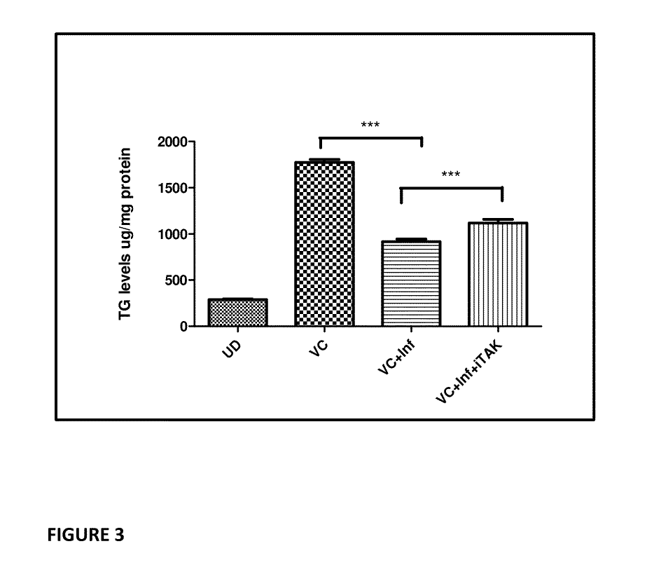

Experiments were performed in which inflammatory cytokines were added to 3T3F442A pre adipocytes (UD) and TG levels were estimated as a measure of adipogenesis. Differentiation was carried out in the presence and absence of TAK-1 inhibitor. Inflammation caused a decrease in TG levels which was partially restored in the presence of TAK-1 inhibitor (FIG. 3). Thus, it can be inferred that TAK-1 inhibition allows efficient storage of fat by enabling formation of new adipocytes.

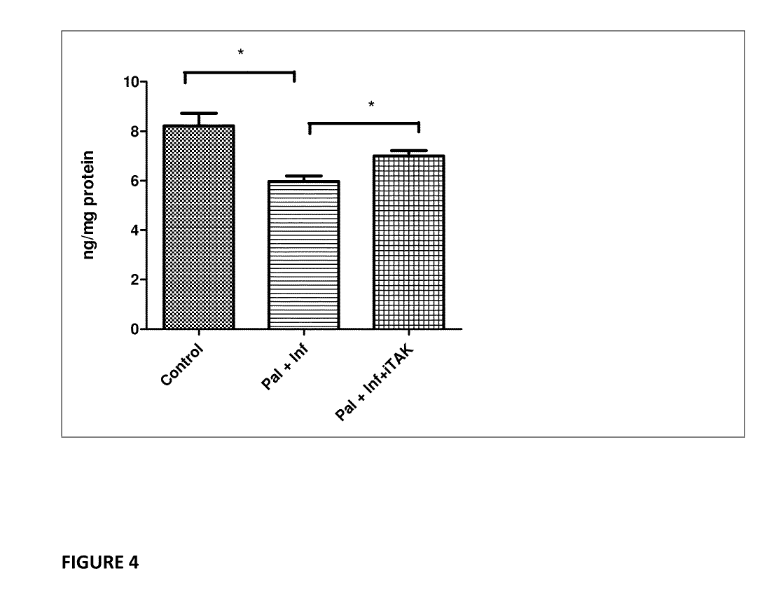

Adiponectin is secreted by insulin sensitive mature adipocytes and positively modulates glucose and lipid metabolism in non adipose tissues such as liver, muscle etc. Increased adiponectin secretion is desirable and is correlated with whole body insulin sensitivity. Inflammation (inf) in the presence of metabolic overload (pal) caused a decrease in adiponectin secretion in 3T3F442A cells. This decrease was partially inhibited by incubation with TAK-1 inhibitor, thus suggesting that TAK-1 inhibition could lead to increased adiponectin levels in vivo (FIG. 4).

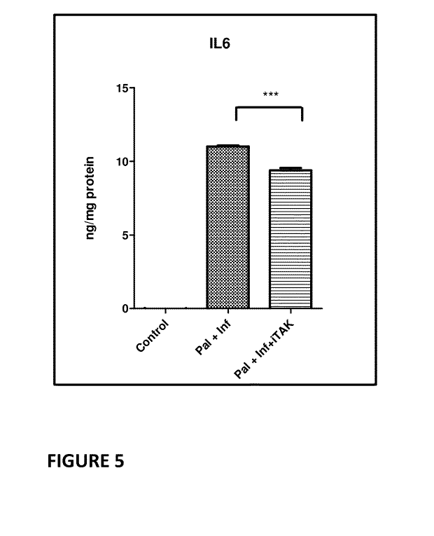

Pro-inflammatory cytokines such as IL6 are released by adipocytes under inflammatory disease conditions. Increased circulating levels of IL6 contribute to insulin resistance, and liver and muscle dysfunction in T2DM. It is thus of interest to decrease release of proinflammatory cytokines from adipose. IL6 secretion in cultured 3T3F442A adipocytes increased in the presence of metabolic overload and inflammation. Inhibition of TAK-1 caused a significant decrease in IL6 secretion (FIG. 5). Thus it was found that TAK-1 inhibition plays a significant role in preventing secretion of pro inflammatory cytokines by adipose tissue in a disease background.

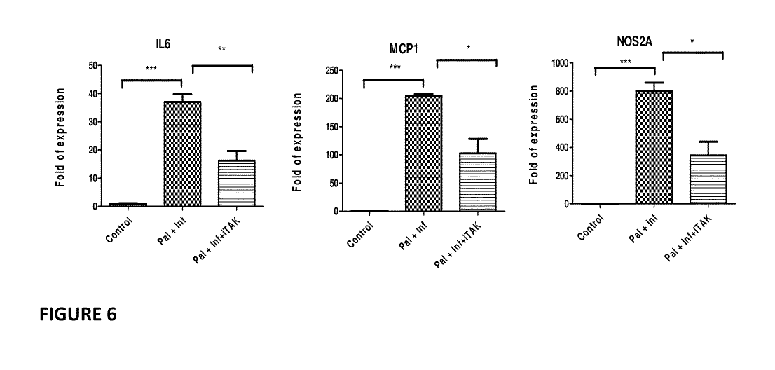

As mentioned above, adipocytes persist in a pro-inflammatory state under pathological conditions. Gene expression profiles for markers such as MCP-1, IL-6 and NOS2A was examined under normal and disease conditions in 3T3F442A. A sharp increase in mRNA levels of these markers was seen under inflammatory conditions, and this increase was significantly attenuated in the presence of TAK-1 inhibition (FIG. 6).

Conclusively inhibition of TAK-1 in adipocytes improve insulin sensitivity and adipocyte function such as adipocgenesis and beneficial adipokine profile by reducing inflammatory and stress signaling

### Impact on Hepatocytes:

Conditions such as steatohepatitis, obesity and type 2 diabetes are associated with an increased production of inflammatory cytokines by the liver. This increase is caused in part by the elevated levels of circulating free fatty acids and intra-hepatic lipid accumulation that are seen in such clinical conditions.

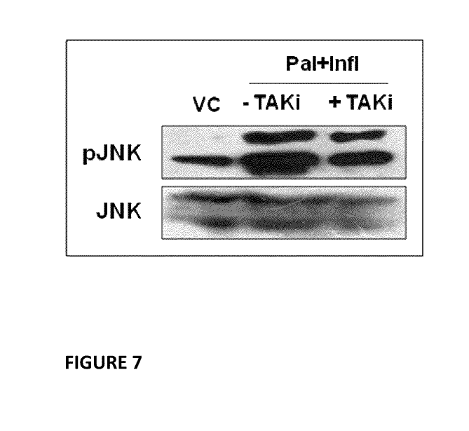

Activation of TAK-1 results in the activation of JNK by mediating the phosphorylation by MAPK kinases. It is known in prior art that activated JNK can directly phosphorylate IRS-1 (insulin receptor substrate-1), thus targeting it for ubiquitin mediated degradation and resulting in the inhibition of insulin signaling. Thus, elevated levels of activated JNK are correlated with the development of insulin resistance in various organs, including the liver. Treatment of hepatoma HepG2 cells with palmitate and inflammatory cytokines resulted in an increase in the phosphorylation of JNK (pJNK) as measured by Western blotting. In this experiment it was observed that inhibition of TAK-1 reduced JNK phosphorylation under the above mentioned condition (FIG. 7). Thus it can be inferred that TAK-1 inhibition reduces JNK phosphorylation thus which in turn results in improved insulin sensitivity.

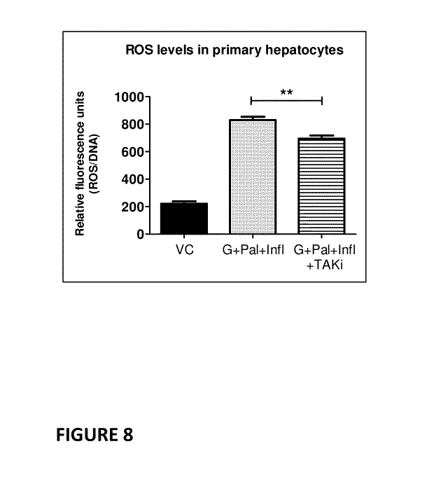

Reactive oxygen species (ROS) are generated as metabolic byproducts, particularly as a byproduct of oxidative phosphorylation. Under normal conditions, the levels of ROS generated are low and tightly controlled, and can function as signaling intermediates. However, metabolic excess and increased inflammation, as seen in steatohepatitis, obesity and T2DM, can result in the over-production of ROS, which in turn can cause cell damage and death by targeting several cellular macromolecules. Treatment of rat primary hepatocytes with glucose (G), palmitate (pal) and inflammatory cytokines (inf) resulted in an increase in intracellular ROS levels. In this experiment it was observed that inhibition of TAK-1 (TAKi) reduced ROS levels under the above mentioned condition (FIG. 8). Thus it can be inferred that TAK-1 inhibition results in a decreasing intracellular ROS levels thus modulating oxidative stress in the liver.

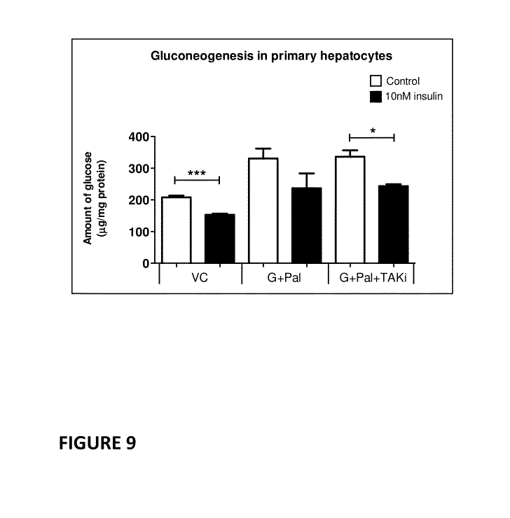

State of hepatocytes in diabetes is characterized by increased basal glucose release, which is in response to metabolic stress and inflammatory signal mediated insulin resistance. Inhibition of TAK-1 by reducing stress and inflammatory signal is identified to moderately improve insulin sensitivity in regulating gluconeogenesis (FIG. 9).

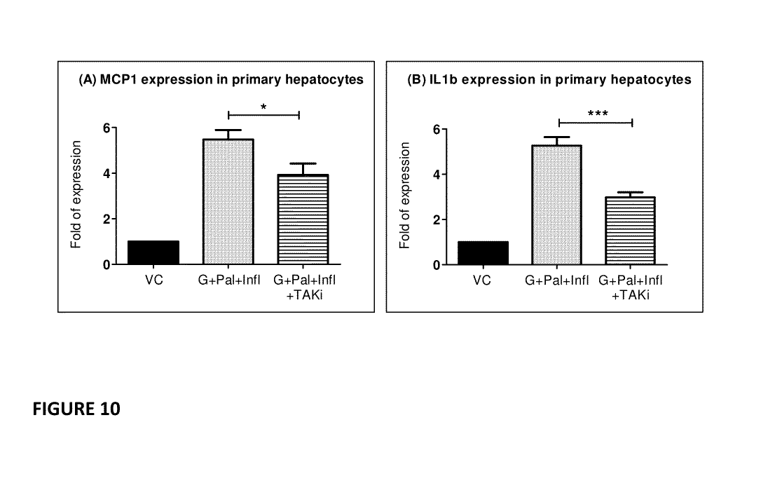

Hepatocytes under diabetic condition are one of the major driver for cardiovascular risk by increasing acute phase proteins in response to inflammatory signaling. This is partly due to increased inflammatory cytokine and chemokine production by hepatocytes in response to metabolic stress and inflammatory response. Inhibition of TAK-1 is shown to reduce chemokine as well as cytokine production in hepatocytes as observed by a statistically significant decrease in the expression of MCP1 and IL-1 beta (IL1b) (FIG. 10).

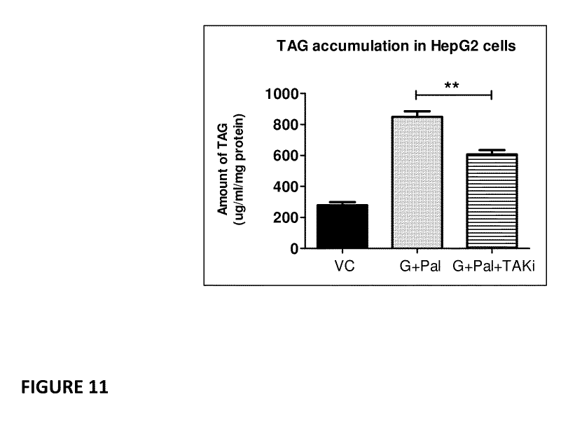

Fatty liver is a critical driver for systemic insulin resistance as it can contribute to basal hyperinsulinemia as well as inflammatory response. Metabolic overload and low grade inflammation can lead to build up of stress signaling mediated increase in liver triglyceride accumulation. Inhibition of TAK-1 is shown to reduce liver triglyceride (TAG) accumulation in rat primary hepatocytes cultured in glucose (G) and palmitate (pal) for 48 hours and hence might be beneficial in improving liver function under diabetes (FIG. 11).

### Impact on Skeletal Muscle:

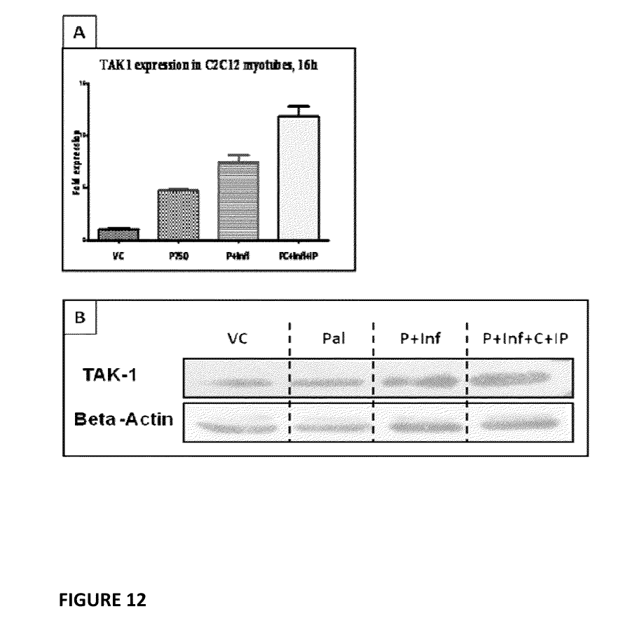

TAK-1 level are increased under patho-physiological conditions. TAK-1 expression as well as protein level was found to be increased in skeletal muscle under pathological condition indicating increased TAK-1 signaling. Metabolic overload (Pal), inflammation (inf), glucocorticoid (C) and beta-adrenergic signaling (IP) up-regulated TAK-1 level (FIG. 12).

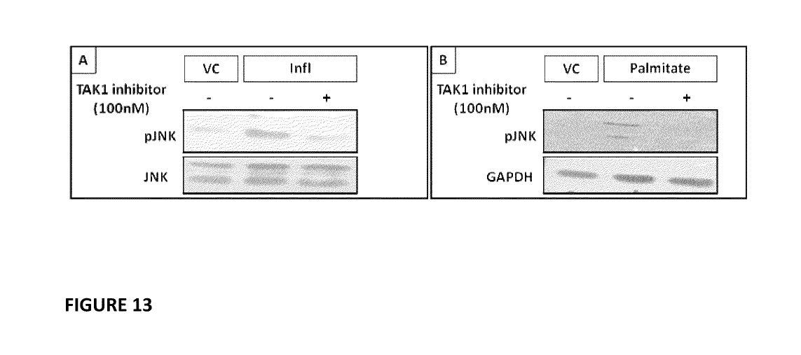

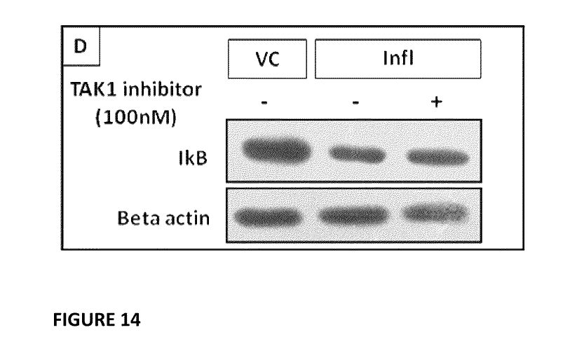

Inhibition of TAK-1 by small molecule inhibitor, (5Z)-7-Oxozeaenol, resulted in decreased activation of JNK in response to inflammatory cytokines (FIG. 13). TAK-1 inhibitor could also reduce activation of JNK in response to metabolic stress (plamitate overload). Moreover, the same inhibitor blunted NFkB pathway by increasing IkB level in cells under inflammatory conditions (Infl) (FIG. 14). Taken together, TAK-1 inhibition could lead to decreased JNK and NFkB signaling under metabolic overload and inflammation.

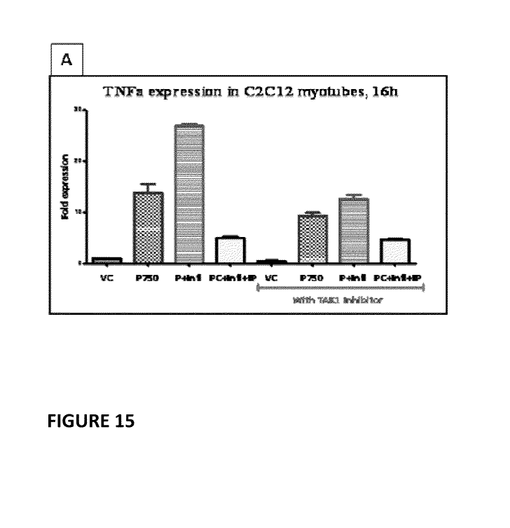

Expression of inflammatory cytokine namely TNFalpha (TNFa) was found to be increased under different patho-physiological conditions. This increase in cytokine gene expression would lead to systemic stress and aggravate metabolic disorder. Since expression of these cytokine is under regulatory control of abovementioned stress kinases, the inventors hypothesized that inhibition of TAK-1 and hence the decreased JNK and NFkB signaling would diminish expression of inflammatory cytokine. In accordance with the hypothesis, down-regulation of TNFa gene expression was observed after TAK-1 inhibition under different pathological conditions involving metabolic overload (Pal) and inflammation (Infl) (FIG. 15). Decrease in inflammatory cytokine gene expression and stress kinases demonstrate the ability of TAK inhibition to handle metabolic overload and inflammation.

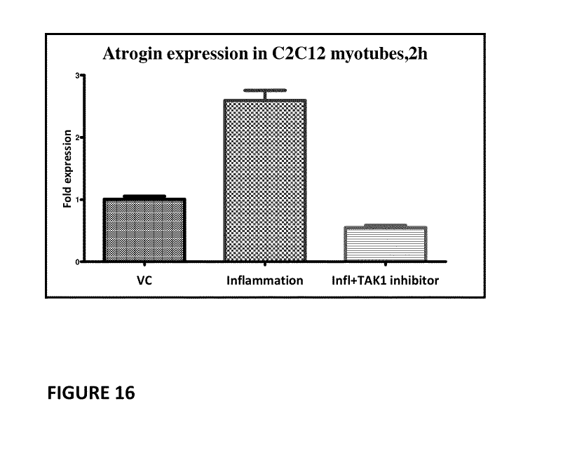

Increased level of inflammation or sepsis shock leads to muscle weakening in humans. Similarly, augmented inflammation and its downstream signaling (JNK and NFkB) increase proteolysis. Muscle weakness is one of the critical factor observed in diabetes that can impact glucose metabolism. An increase in marker of muscle proteolysis (Atrogin) was observed by inflammatory cytokines (Inf I) which was reversed by TAK-1 inhibition. Furthermore, TAK-1 inhibition could restrain skeletal muscle proteolysis under different patho-physiological conditions. (FIG. 16)

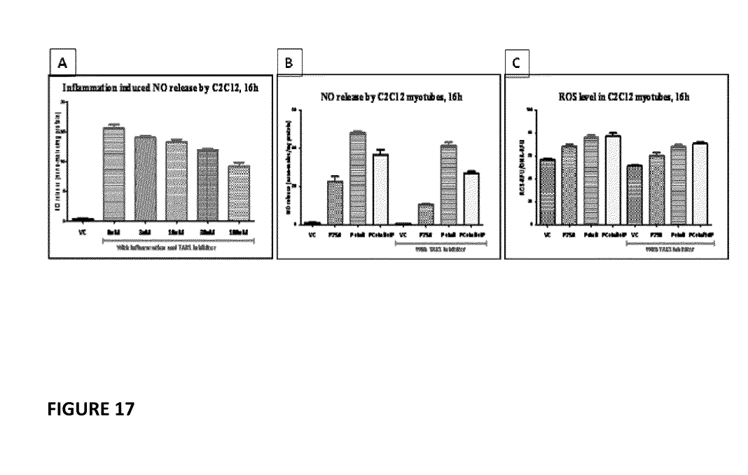

Oxidative stress mediated by both ROS and RNS (NO) is known to impair mitochondrial function and induced insulin resistance in skeletal muscle (FIG. 17). Furthermore, it is also known that metabolic overload and inflammatory signal drives oxidative stress in skeletal muscles. From the experiments conducted it was observed that TAK-1 inhibitor reduced inflammation induced NO release from C2C12 myotubes in a dose dependent manner (FIG. 17 A). Similarly, it reduced NO release in skeletal muscles under metabolic overload (high glucose and palmitate) and inflammatory conditions (FIG. 17 B). In consistent with NO, ROS levels were also reduced by TAK-1 inhibitor under different disease conditions (FIG. 17 C).

### Impact on Mitochondria

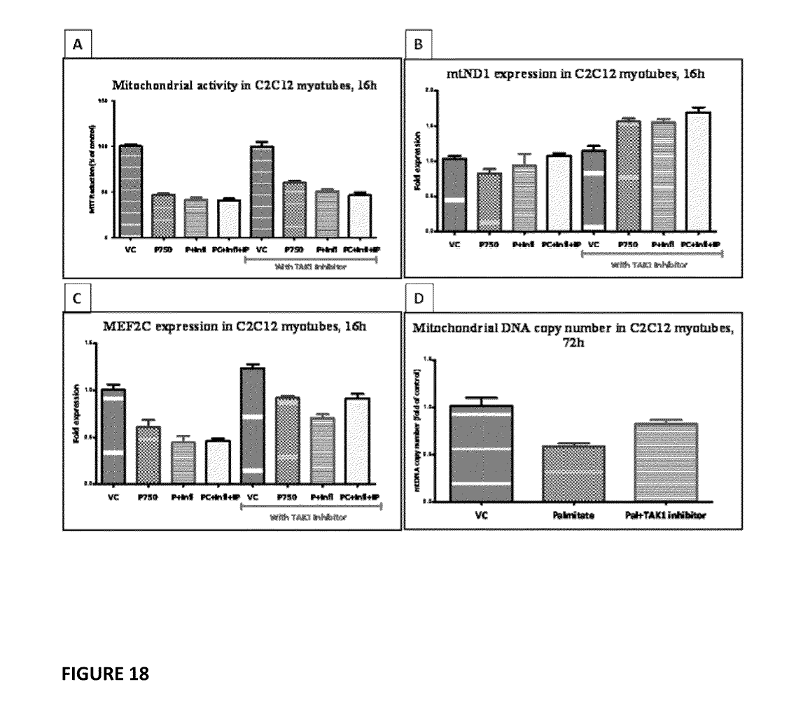

Increased level of inflammation and oxidative stress under different pathological conditions can cause mitochondrial damage and hence can reduce mitochondrial activity. In fact, decreased mitochondrial activity was observed under these conditions as measured by reduction of MTT. In consistent with TAK-1 inhibitor's ability to decrease inflammation and oxidative stress, it could increase mitochondrial activity of muscle cells under similar conditions. Likewise, TAK-1 inhibition could up-regulate the expression of mitochondrial genes such as mtND1. Moreover, TAK-1 inhibition resulted in increased mitochondrial DNA copy number along with muscle enriched transcription factor (MEF2C) under conditions of metabolic overload or inflammation. Therefore, the inventors concluded that TAK-1 inhibition can increase mitochondrial biogenesis and increased activity (FIG. 18).

### Impact on Glucose and Fat Metabolism

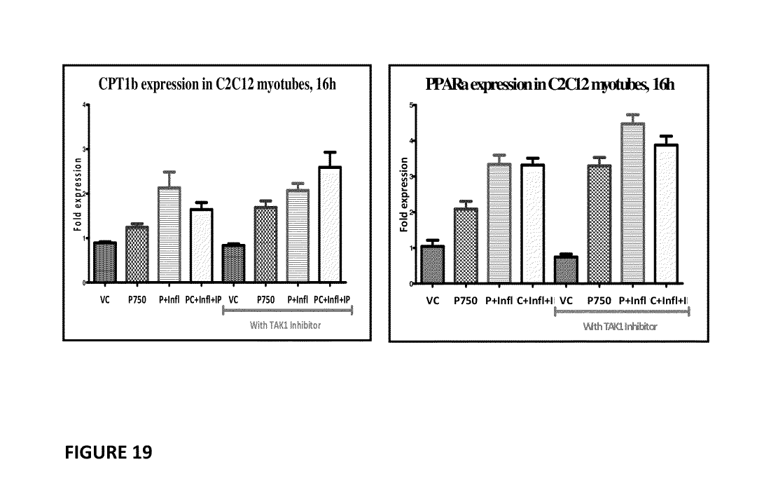

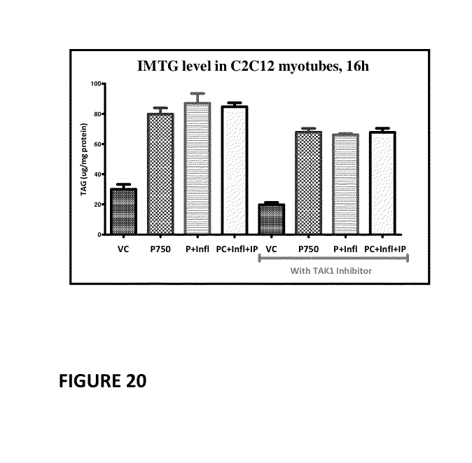

In consistent with mitochondrial biogenesis, an increase in fatty acid oxidation was observed by TAK-1 inhibition under different pathological conditions as measured by expression of PPARa and CPT1b (FIG. 19). Increased fatty acid oxidation by TAK-1 inhibition was coupled with decreased IMTG storage under similar pathological conditions (FIG. 20). Hence, the inventors concluded that TAK-1 inhibition regulates fat metabolism by increasing fat burning and by decreasing storage in skeletal muscle.

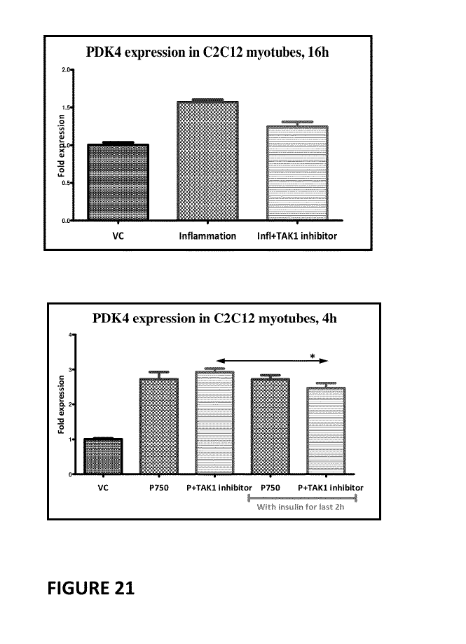

Augmented inflammatory and stress signal reduces glucose oxidation by increasing PDK4. Moreover, it is known that insulin treatment can reduce PDK4 expression in insulin sensitive cells but not in insulin resistance cells. Insulin was failed to decrease PDK4 expression under inflammatory cytokines (Infl) and/or metabolic overload (P750) condition. However, TAK-1 inhibition along with these conditions enabled the cells to respond to insulin and thus a decrease in PDK4 expression was observed (FIG. 21). The experiments showed that a slight decrease in pathological conditions induced PDK4 expression was observed suggesting more glucose utilization through TCA cycle.

Taken together, the experimental results indicate that TAK-1 inhibition increases muscle glucose utilization under different pathological condition.

### Increased Insulin Sensitivity by TAK-1 Inhibition

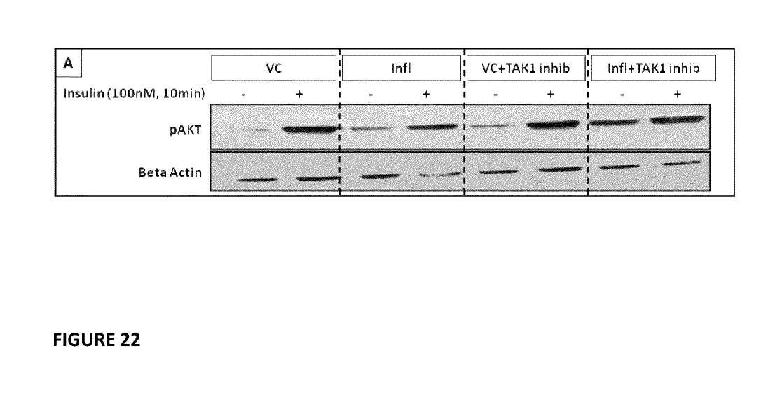

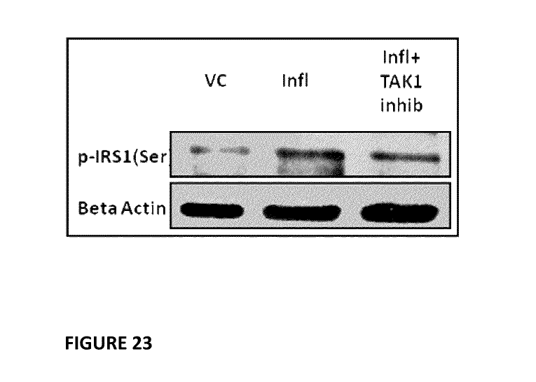

The impact of TAK-1 inhibition on muscle insulin resistance caused by increased inflammation was measured. Increased inflammation (Infl) could lead to diminished activation of AKT in response to insulin. Activation of AKT was restored by TAK-1 inhibition under similar condition (FIG. 22). Moreover, inflammation (Infl) induced increased level of phosphorylated IRS1 at serine residue (a modification required for insulin resistance) was diminished by TAK-1 inhibition (FIG. 23). Hence, TAK-1 inhibition was found to reduce muscle insulin resistance.

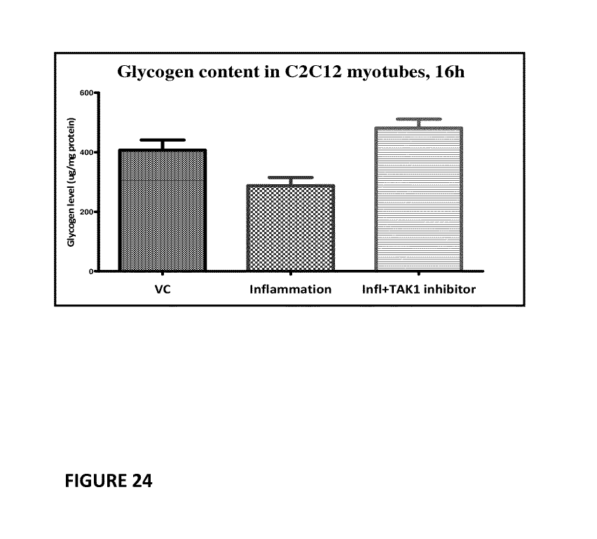

Similarly, insulin was unable to increase muscle glycogen content under inflammatory (Infl) condition whereas TAK-1 inhibition could bring about insulin mediated increase in glycogen content (FIG. 24).

Conclusively, TAk-1 inhibition improves mitochondrial content, insulin sensitivity and glucose oxidation in skeletal muscle which will be ideal for an antidiabetic treatment.

### Beta Cells

Metabolic overload and systemic inflammatory response mediated activation of stress signals is a hallmark of metabolic disorders and can lead to impact loss of glucose sensitivity in beta cells and thereby impact insulin secretion in diabetes.

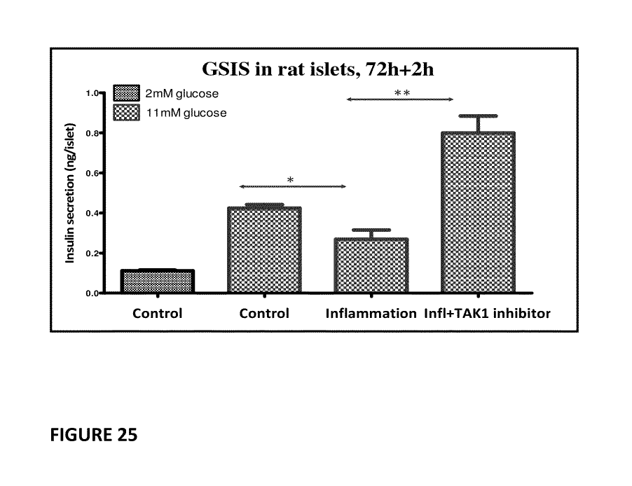

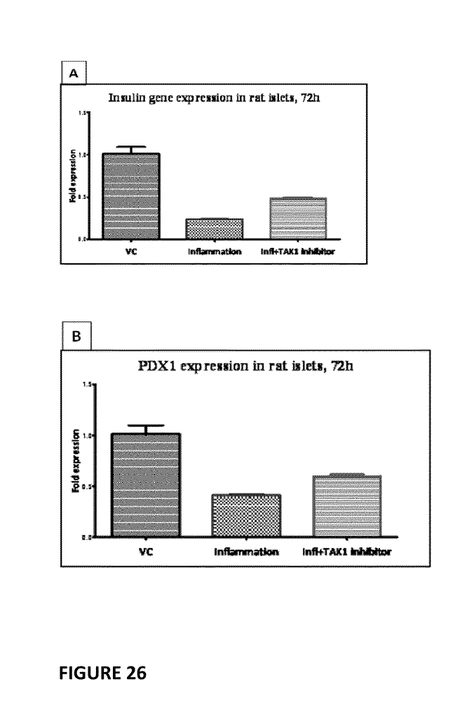

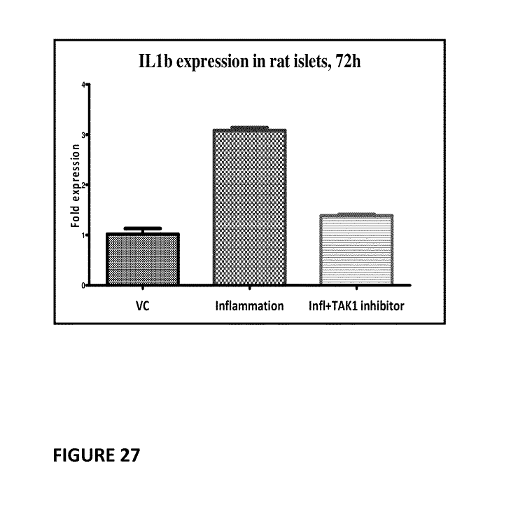

Exposure of rat islets to inflammatory condition (Infl) blunted glucose stimulated insulin secretion (GSIS). Inhibition of TAK-1 in such condition restored GSIS (FIG. 25). Moreover, TAK-1 inhibitor could up-regulate insulin gene as well as PDX1 expression which was blocked under inflammation (FIG. 26). Furthermore, TAK-1 inhibitor could reduce inflammation induced IL1b expression in rat islet indicating TAK-1 inhibitors potency to prevent inflammation induced damage (FIG. 27). Taken together, inhibition of TAK-1 under inflammatory condition can increase GSIS and beta cell insulin content while up-regulating beta cell health $ proliferation marker-PDX1, and down-regulating inflammatory cytokine (IL1b) gene expression.

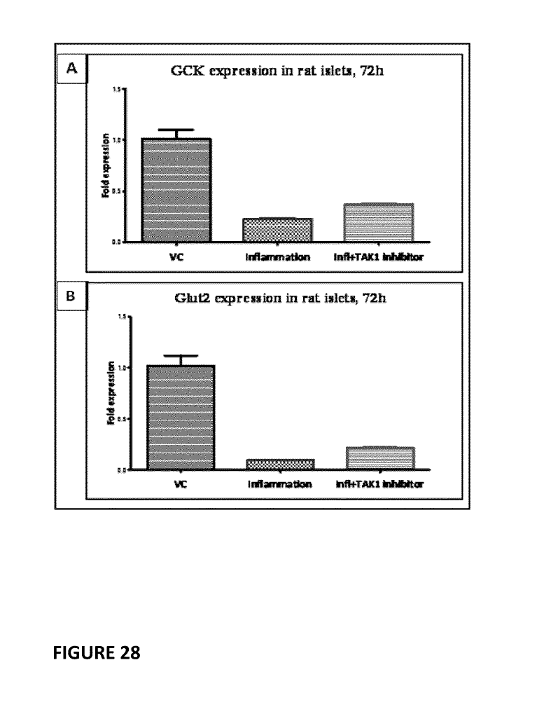

Inhibition of TAK-1 could also increase beta cell glucose sensing machinery comprising of glucokinase (GCK) and Glut2 (slc2a2) (FIG. 28). Therefore, targeting TAK-1 will have long term beneficial impact in improving beta cell health as well as function under diabetic conditions

### Impact on Macrophages

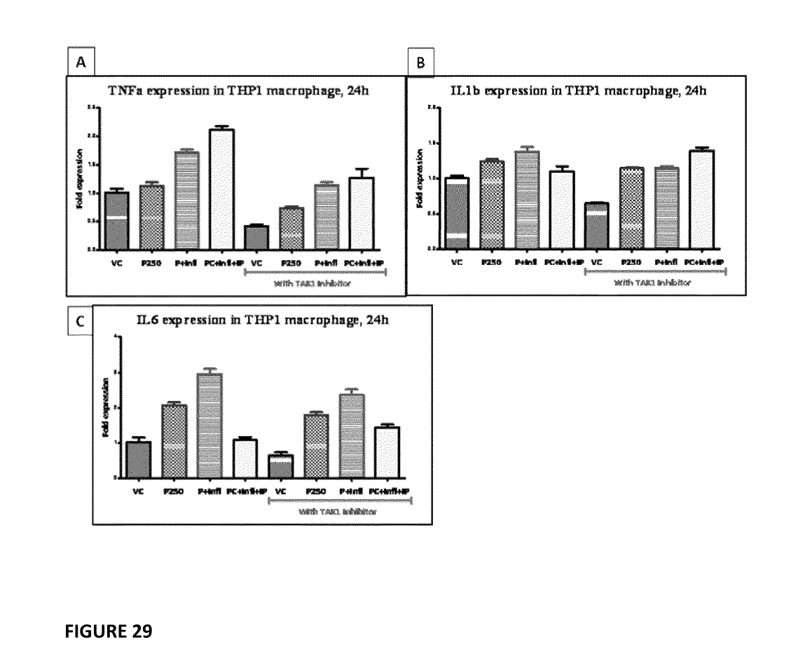

Obese insulin resistance is characterized by low grade inflammatory condition. Several studies demonstrate that activation state of macrophages along with infiltration of the other innate and adaptive immune cells, contribute to augmentation of insulin resistance in this phenotype. In multiple tissues, especially in adipose tissue, macrophages has a pro-inflammatory, classical phenotype which increases the production of inflammatory cytokine (Infl) such as TNF, IL6 etc which are the major drive for observed metabolic dysregulation in key metabolic tissues. Inhibiting TAK-1 in THP1 macrophage cells reduced the expression of proinflammatory cytokines such as TNF, IL6 (FIG. 29).

### In Vivo Experiments

### Fasting Glucose Levels

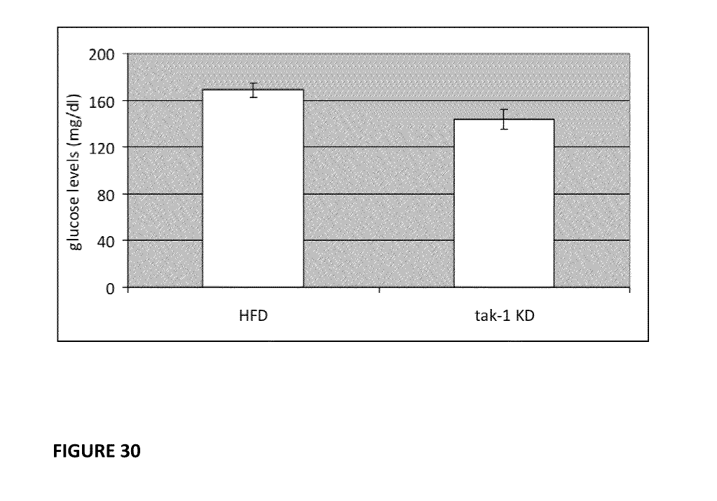

Mice were fed on high fat diet for 12 weeks and divided into two groups. For one group, DNA construct carrying ShRNA for tak-1 gene was delivered to liver and muscle (tak-1 KD), and the control group received plasmid that does not harbor tak-1 ShRNA (HFD). DNA was delivered every 15 days for 8 weeks. Mice were fasted for 16 hours and the glucose levels were measured (FIG. 30). A substantial decrease was seen in the fasting glucose levels in the TAK 1 Knockdown model. All values are mean±SEM (n=7), p=0.019 students t test (unpaired).

### Glucose Tolerance

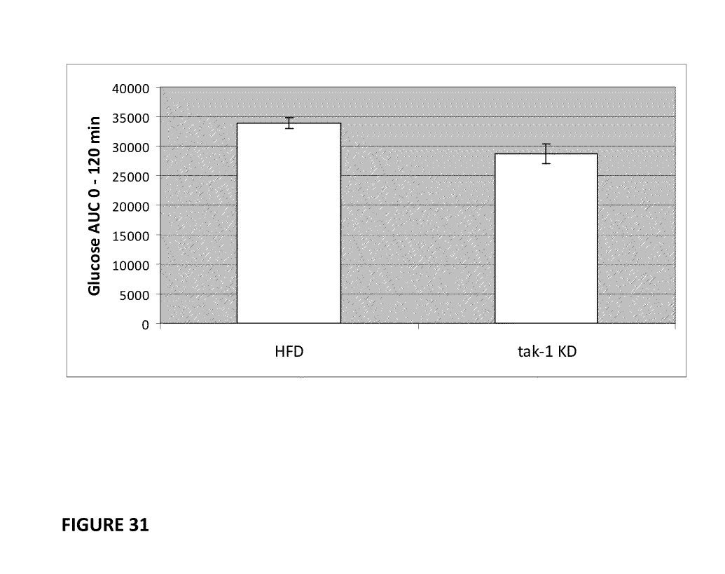

Mice were fed on high fat diet for 12 weeks and divided into two groups. For one group, DNA construct carrying ShRNA for tak-1 gene was delivered to liver and muscle (tak-1 KD), and the control group received plasmid that does not harbor tak-1 ShRNA (HFD). DNA was delivered every 15 days for 8 weeks. Mice were fasted for 6 hours and oral glucose tolerance was measured after dosing 2 g/kg bodyweight of glucose to all the mice. (FIG. 31). A significant improvement in glucose tolerance was seen in the tak-1 KD compared to the HFD model. All values are mean±SEM (n=8) p=0.02 students t test (unpaired).

### Insulin Sensitivity

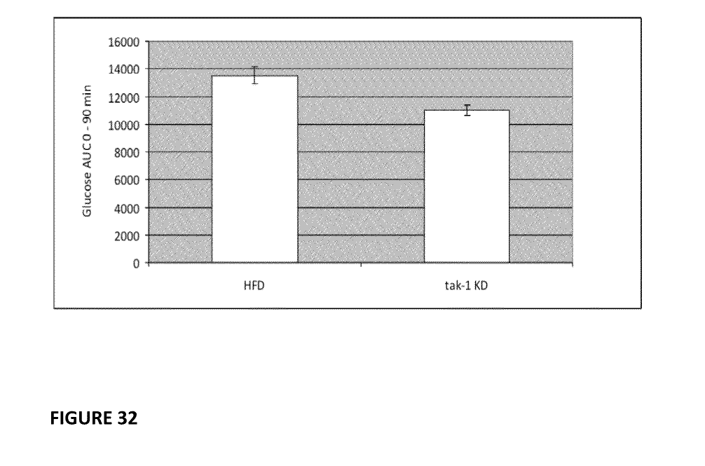

Mice were fed on high fat diet for 12 weeks and divided into two groups. For one group, DNA construct carrying ShRNA for tak-1 gene was delivered to liver and muscle (tak-1 KD), and the control group received plasmid that does not harbor tak-1 ShRNA (HFD). DNA was delivered every 15 days for 8 weeks. Mice were fasted for 6 hours and glucose levels were measured at various time points after insulin dosing (1 lU/kg bodyweight, dosed intra peritoneally) (FIG. 32). A significant improvement in insulin sensitivity was seen in the tak-1 KD compared to the HFD model. All values are mean±SEM (n=8), p=0.012 students t test (unpaired).

### Serum Triglyceride

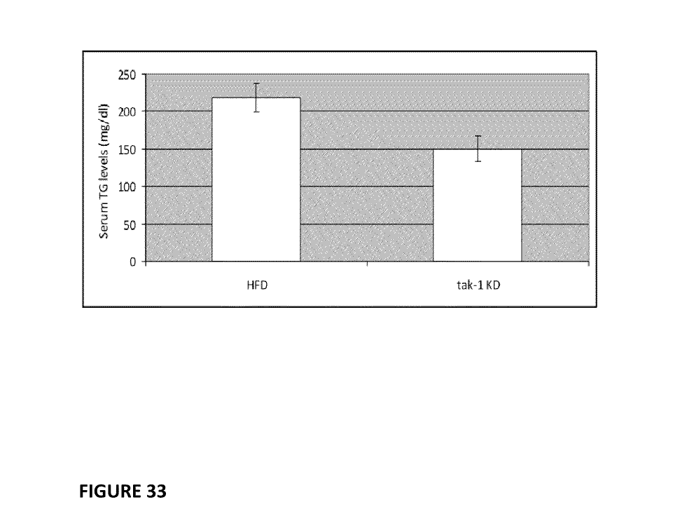

Mice were fed on high fat diet for 12 weeks and divided into two groups. For one group, DNA construct carrying ShRNA for tak-1 gene was delivered to liver and muscle (tak-1 KD), and the control group received plasmid that does not harbor tak-1 ShRNA (HFD). DNA was delivered every 15 days for 8 weeks. Mice were fasted for 12 hours and serum triglyceride levels were measured (FIG. 33) A significant decrease in serum triglyceride level was seen in the tak-1 KD compared to the HFD model All values are mean±SEM (n=7), p=0.02 students t test (unpaired).

It is understood that the foregoing detailed description and accompanying examples are merely illustrative and are not to be taken as limitations upon the scope of the invention, which is defined solely by the appended claims and their equivalents. Various changes and modifications to the disclosed aspects will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

1. A method of using inhibitors of TAK-1 in preparing a medicament for therapy of diseases which are based on loss of glycemic control in mammals.

2. The method of use according to claim 1, wherein the medicament is used for the prevention or alleviation of pathological abnormalities of the metabolism of mammals such as hyperglycemia, low glucose tolerance, low insulin sensitivity, glucosuria, hyperlipidemia, hypertriglyceridemia, hypercholesterolemia, dyslipedemia, metabolic acidosis, obesity, diabetes mellitus and diabetes related secondary complications.

3. The method of use according to claim 1, characterized in that inhibitors, pseudosubstrates, inhibitors of TAK-1 expression, binding proteins, or antibodies against the enzyme proteins or combination of the designated inhibitors of TAK-1 or TAK-1 like enzyme activity.

4. A method for lowering elevated blood glucose levels in mammals comprising administering at least one oral administration of a therapeutically effective amount of at least one inhibitor of TAK-1 or of TAK-1 enzyme activity.

5. The method according to claim 4, wherein the at least one inhibitor is administered in combination with at least one carrier substance.

6. The method according to claim 4, wherein the at least one inhibitor is administered in multiple administrations

7. The method according to claim 4, wherein the mammals demonstrate at least one of clinically appropriate basal, fasting and post-prandial hyperglycemia.

8. The method according to claim 4, wherein the administration is for the prevention or alleviation of pathological abnormalities of metabolism of mammals such as diabetes, hyperglycemia, low glucose tolerance, glucosuria, metabolic acidosis, hyperinsulinemia, hyperlipidemia, hypertriglyceridemia, hypercholesterolemia, dyslipidemia, obesity and diabetic related secondary complications.

9. The method according to claim 4, wherein the method comprises administering at least one oral administration of a therapeutically effective amount of at least one inhibitor of TAK-1 or of TAK-1 enzyme activity in combination with an adjuvant selected from and not limited to:
(a) dipeptidyl peptidase-IV (DP-IV) inhibitors;
(b) insulin sensitizing agents;
(c) insulin and insulin mimetics;
(d) sulfonylureas and other insulin secretagogues;
(e) alpha.-glucosidase inhibitors; and
(f) GLP-1, GLP-1 analogs, and GLP-1 receptor agonists.

10. The method according to claim 9, wherein the adjuvant is a biguanide.

11. The method according to claim 10, wherein the biguanide is metformin.
